SEQUENCE LISTING (14 sequences)

SEQ ID NO: 1 - protein, 466 aa, Corynebacterium glutamicum
MKNKRVAIIGAGPSGIAQLRAFESAEKQGHEIPELVCFEKQDTWGGQWNYSWRTGTDSYGEPVHSSMYRNLWSNGPKEVLEFAEYSFDEHFGKPISSYPPREVLWDYIAGRAKKSNVEKYIKFAHVVRWVSFDEATKLFTVTVENLRTGETSSDTYDNVIVGAGHFSFPNVPHFDGVETFPGQIVHAHEFRGAEAVADKDILLIGASYSAEDIGTQAYKMGARSVTFSYRSNPMGYEWPEEMTELPLVERFDGSEVHFVNGEKRKVDIVVFCTGYLHHYPFMPSELTLSSPNNLYPDTLYRGVVSEANNQLFWLGAQDQWLTFNMFDAQAWYVRDVILGRVALPSKEAQRNHMDKWLSRFEGLKSENDQIDFQCDYVEDLIDQTDYPSFDLKEVANILKGWVKSKEEDILNYRDYTYTSVMTGTTSVEHHTPWMIELDDSLERYLSEPQEDEARQVYRGKKVRDKA

SEQ ID NO: 2 - protein, 466 aa, Corynebacterium glutamicum
MKNKRVAIIGAGPSGIAQLRAFESAEKQGHEIPELVCFEKQDTWGGQWNYSWRTGTDSYGEPVHSSMYRNLWSNGPKEVLEFAEYSFDEHFGKPISSYPPREVLWDYIAGRAKKSNVEKYIKFAHVVRWVSFDEATKLFTVTVENLRTGETSSDTYDNVIVGAGHFSFPNVPHFDGVETFPGQIMHAHEFRGAEAVADKDILLIGASYSAEDIGTQAYKMGARSVTFSYRSNPMGYEWPEEMTELPLVERFDGSEVHFVNGEKRKVDIVVFCTGYLHHYPFMPSELTLSSPNNLYPDTLYRGVVSEANNQLFWLGAQDQWLTFNMFDAQAWYVRDVILGRVALPSKEAQRNHMDKWLSRFEGLKSENDQIDFQCDYVEDLIDQTDYPSFDLKEVANILKGWMKSKEEDILNYRDYTYTSVMTGTTSVEHHTPWMIELDDSLERYLSEPQEDEARQVYRGKKVRDKA

SEQ ID NO: 3 - protein, 466 aa, Corynebacterium glutamicum
MKNKRVAIIGAGPSGIAQLRAFESAEKQGHEIPELVCFEKQDTWGGQWNYSWRTGTDSYGEPVHSSMYRNLWSNGPKEVLEFAEYSFDEHFGKPISSYPPREVLWDYIAGRAKKSNVEKYIKFAHVVRWVSFDEATKLFTVTVENLRTGETSSDTYDNVIVGAGHFSFPNVPHFDGVETFPGQIVHAHEFRGAEAVADKDILLIGASYSAEDIGTQAYKMGARSVTFSYRSNPMGYEWPEEMTELPLVERFDGSEVHFVNGEKRKVDIVVFCTGYLHHYPFMPSELTLSSPNNLYPDTLYRGVVSEANNQLFWLGAQDQWLTFNMFDAQAWYVRDVILGRVALPSKEAQRNHMDKWLSRFEGLKSENDQIDFQCDYVEDLIDQTDYPSFDLKEVANILKGWMKSKEEDILNYRDYTYTSVMTGTTSVEHHTPWMIELDDSLERYLSEPQEDEARQVYRGKKVRDKA

SEQ ID NO: 4 - DNA, 32 bp, synthetic construct
  misc_feature 1..32: note = synthetic primer
  source 1..32: mol_type = other DNA
ccaggtcagatcgtgcatgctcacgagttccg

SEQ ID NO: 5 - DNA, 32 bp, synthetic construct
  misc_feature 1..32: note = synthetic primer
  source 1..32: mol_type = other DNA
ctcgtgagcatgcacgatctgacctgggaaag

SEQ ID NO: 6 - DNA, 32 bp, synthetic construct
  misc_feature 1..32: note = synthetic primer
  source 1..32: mol_type = other DNA
ttgaagggctggatgaagtcgaaggaggagga

SEQ ID NO: 7 - DNA, 35 bp, synthetic construct
  misc_feature 1..35: note = synthetic primer
  source 1..35: mol_type = other DNA
ctccttcgacttcatccagcccttcaagatattcg

SEQ ID NO: 8 - DNA, 55 bp, synthetic construct
  misc_feature 1..55: note = synthetic primer
  source 1..55: mol_type = other DNA
ttcattaaagaggagaaaggtacatggagatggttatgaagaataagcgcgttgc

SEQ ID NO: 9 - DNA, 50 bp, synthetic construct
  misc_feature 1..50: note = synthetic primer
  source 1..50: mol_type = other DNA
catcaccatcaccatcacggatccgaaaacatgaagaataagcgcgttgc

SEQ ID NO: 10 - DNA, 55 bp, synthetic construct
  misc_feature 1..55: note = synthetic primer
  source 1..55: mol_type = other DNA
gagcctttcgttttatttgatgcctctagattaggctttatcgcggactttcttg

SEQ ID NO: 11 - DNA, 52 bp, synthetic construct
  misc_feature 1..52: note = synthetic primer
  misc_feature 28..29: note = n is a, c, g, or t
  misc_feature 30: note = k is g or t
  source 1..52: mol_type = other DNA
ggtgtggagactttcccaggtcagatcnnkcatgctcacgagttccgtggtg

SEQ ID NO: 12 - DNA, 52 bp, synthetic construct
  misc_feature 1..52: note = synthetic primer
  misc_feature 28: note = m is a or c
  misc_feature 29..30: note = n is a, c, g, or t
  source 1..52: mol_type = other DNA
gttgaggatatcctcctccttcgacttmnnccagcccttcaagatattcgcg

SEQ ID NO: 13 - protein, 466 aa, Corynebacterium glutamicum
MKNKRVAIIGAGPSGIAQLRAFESAEKQGHEIPELVCFEKQDTWGGQWNYSWRTGTDSYGEPVHSSMYRNLWSNGPKEVLEFAEYSFDEHFGKPISSYPPREVLWDYIAGRAKKSNVEKYIKFAHVVRWVSFDEATKLFTVTVENLRTGETSSDTYDNVIVGAGHFSFPNVPHFDGVETFPGQIMHAHEFRGAEAVADKDILLIGASYSAEDIGTQAYKMGARSVTFSYRSNPMGYEWPEEMTELPLVERFDGSEVHFVNGEKRKVDIVVFCTGYLHHYPFMPSELTLSSPNNLYPDTLYRGVVSEANNQLFWLGAQDQWLTFNMFDAQAWYVRDVILGRVALPSKEAQRNHMDKWLSRFEGLKSENDQIDFQCDYVEDLIDQTDYPSFDLKEVANILKGWRKSKEEDILNYRDYTYTSVMTGTTSVEHHTPWMIELDDSLERYLSEPQEDEARQVYRGKKVRDKA

SEQ ID NO: 14 - protein, 466 aa, Corynebacterium glutamicum
MKNKRVAIIGAGPSGIAQLRAFESAEKQGHEIPELVCFEKQDTWGGQWNYSWRTGTDSYGEPVHSSMYRNLWSNGPKEVLEFAEYSFDEHFGKPISSYPPREVLWDYIAGRAKKSNVEKYIKFAHVVRWVSFDEATKLFTVTVENLRTGETSSDTYDNVIVGAGHFSFPNVPHFDGVETFPGQILHAHEFRGAEAVADKDILLIGASYSAEDIGTQAYKMGARSVTFSYRSNPMGYEWPEEMTELPLVERFDGSEVHFVNGEKRKVDIVVFCTGYLHHYPFMPSELTLSSPNNLYPDTLYRGVVSEANNQLFWLGAQDQWLTFNMFDAQAWYVRDVILGRVALPSKEAQRNHMDKWLSRFEGLKSENDQIDFQCDYVEDLIDQTDYPSFDLKEVANILKGWAKSKEEDILNYRDYTYTSVMTGTTSVEHHTPWMIELDDSLERYLSEPQEDEARQVYRGKKVRDKA

(19) United States
(12) Patent Application Publication
(10) Pub. No.: US 2023/0279367 A1
(43) Pub. Date: Sep. 7, 2023

(54) METHOD AND RECOMBINANT POLYPEPTIDE FOR INCREASING PRODUCTION OF INDIGOID COMPOUND

(71) Applicant: National Tsing Hua University, Hsinchu (TW)

(72) Inventors: Roa-Pu Claire Shen, Hsinchu (TW); Zi-Yi Yang, Hsinchu (TW); Vincent Ruan, Hsinchu (TW); Pi-Ju Yang, Hsinchu (TW)

(21) Appl. No.: 18/076,680

(22) Filed: Dec. 7, 2022

(30) Foreign Application Priority Data
Mar. 3, 2022 (TW) 111107819

(51) Int. Cl. C12N 9/02; C12N 1/20

(52) U.S. Cl. CPC C12N 9/0071; C12N 1/205; C12Y 114/13008; C12R 2001/19

(57) ABSTRACT

The present disclosure provides a method for increasing production of an indigoid compound, including the following steps: (a) mutating a wild-type flavin-containing monooxygenase to a mutant flavin-containing monooxygenase expressed in Escherichia coli; and (b) culturing the Escherichia coli in a bacterial culture medium comprising tryptophan to allow the mutant FMO to interact with tryptophan for a predetermined time to convert the tryptophan into the indigoid compound. Compared to the wild-type flavin-containing monooxygenase, the mutant flavin-containing monooxygenase increases production of the indigoid compound. The present disclosure also provides a recombinant polypeptide for increasing production of the indigoid compound.

### CROSS-REFERENCE TO RELATED APPLICATION

This application claims priority of Taiwan patent application No. 111107819, filed on Mar. 3, 2022, the content of which is incorporated herein in its entirety by reference.

### STATEMENT REGARDING SEQUENCE LISTING

The sequence listing associated with this application is provided in text format in lieu of a paper copy and is hereby incorporated by reference into the specification. The name of the XML file containing the sequence listing is 110F0621-IE Sequence listing. The XML file is 21000 bytes; was created on Nov. 2, 2022.

### BACKGROUND OF THE INVENTION

### 1. Field of the Invention

The present invention relates to a method and a recombinant polypeptide for increasing production of an indigoid compound.

### 2. The Prior Art

Indigoid compounds are a group of colored aromatic ring biochemicals derived from indole. Oxygenase converts the precursor indole into 3-hydroxyindole and 2-hydroxyindole. 2-hydroxyindole and 3-hydroxyindole spontaneously dimerize to form indirubin, while two molecules of 3-hydroxyindole spontaneously dimerize to form indigo (see FIG. 1). The oxygenase commonly used in the prior art includes naphthalene dioxygenase (NDO) and flavin-containing monooxygenase (FMO). Since the primary reactants are not indole, the synthesis efficiency is mostly not good.

In addition, due to the fact that most of the current oxygenase region hydroxylation selectively tends to generate 3-hydroxyindole, so that most of the genetically modified strains only produce indigo, indirubin production is quite difficult. Both indigo and indirubin are natural plant metabolic chemicals, both have anti-inflammatory properties and can be used as cosmetics, food, dyes of textiles and health care foods. Indirubin is one of the main medicinal ingredients in Angelica Longhui Pills commonly used in traditional Chinese medicine. It is used to treat chronic diseases such as chronic myeloid leukemia, and its derivatives have the potential to inhibit cancer and treat Alzheimer's disease. Therefore, those skilled in the art are actively working on developing a method for increasing production of indigo and indirubin.

In order to solve the above-mentioned problems, those skilled in the art urgently need to develop a novel method for increasing production of an indigoid compound (e.g., indigo and indirubin) for the benefit of a large group of people in need thereof.

### SUMMARY OF THE INVENTION

A primary objective of the present invention is to provide a method for increasing production of an indigoid compound, comprising the following steps: (a) mutating a wild-type flavin-containing monooxygenase (FMO) to a mutant FMO expressed in Escherichia coli; and (b) culturing the Escherichia coli in a bacterial culture medium comprising tryptophan to allow the mutant FMO to interact with tryptophan for a predetermined time to convert the tryptophan into the indigoid compound; wherein compared to the wild-type FMO, the mutant FMO increases production of the indigoid compound.

According to an embodiment of the present invention, both the wild-type FMO and the mutant FMO are from Corynebacterium glutamicum.

According to an embodiment of the present invention, the mutant FMO comprises at least one mutation site.

According to an embodiment of the present invention, the at least one mutation site is located at amino acid residue 185 of the mutant FMO, amino acid residue 402 of the mutant FMO, or a combination thereof.

According to an embodiment of the present invention, the amino acid residue 185 of the mutant FMO is formed by mutating methionine residue 185 of the wild-type FMO to a valine residue.

According to an embodiment of the present invention, the amino acid residue 402 of the mutant FMO is formed by mutating valine residue 402 of the wild-type FMO to a methionine residue.

According to an embodiment of the present invention, the amino acid residue 402 of the mutant FMO is formed by mutating valine residue 402 of the wild-type FMO to the arginine residue.

According to an embodiment of the present invention, the amino acid residue 185 of the mutant FMO is formed by mutating methionine residue 185 of the wild-type FMO to a leucine residue, and the amino acid residue 402 of the mutant FMO is formed by mutating valine residue 402 of the wild-type FMO to an alanine residue.

According to an embodiment of the present invention, the indigoid compound is selected from the group consisting of: indigo, indirubin, and a combination thereof.

According to an embodiment of the present invention, the bacterial culture medium comprising tryptophan further comprises cysteine.

According to an embodiment of the present invention, the mutant FMO is expressed in Escherichia coli XL-1 Blue.

According to an embodiment of the present invention, in step (b), the predetermined time is at least 24 hours.

Another objective of the present invention is to provide a recombinant polypeptide for increasing production of an indigoid compound, comprising an amino acid sequence selected from the group consisting of: SEQ ID NO:1, SEQ ID NO:2, SEQ ID NO:3, SEQ ID NO:13, and SEQ ID NO:14.

According to an embodiment of the present invention, the recombinant polypeptide is a polypeptide of a mutant FMO.

According to an embodiment of the present invention, the amino acid sequence of SEQ ID NO:1 is formed by mutating methionine residue 185 of a wild-type FMO to a valine residue.

According to an embodiment of the present invention, the amino acid sequence of SEQ ID NO:2 is formed by mutating valine residue 402 of a wild-type FMO to a methionine residue.

According to an embodiment of the present invention, the amino acid sequence of SEQ ID NO:3 is formed by mutating methionine residue 185 of a wild-type FMO to a valine residue, and by mutating valine residue 402 of the wild-type FMO to a methionine residue.

According to an embodiment of the present invention, the amino acid sequence of SEQ ID NO:13 is formed by mutating valine residue 402 of the wild-type FMO to the arginine residue.

According to an embodiment of the present invention, the amino acid sequence of SEQ ID NO:14 is formed by mutating methionine residue 185 of a wild-type FMO to a leucine residue, and by mutating valine residue 402 of the wild-type FMO to an alanine residue.

In summary, the present invention has the following effects. The mutant FMO can be screened out by using random mutation technology and screening of enzymes through protein engineering technology, and it is found that the combination of amino acid residues at specific positions in the amino acid sequences of the mutant FMO can effectively increase production of indigoid compounds, so that the yield of indigo and indirubin is more than 2 to 3 times higher than that of strains expressing wild-type FMO.

### BRIEF DESCRIPTION OF THE DRAWINGS

The following drawings form part of the present specification and are included here to further demonstrate some aspects of the present invention, which can be better understood by reference to one or more of these drawings, in combination with the detailed description of the embodiments presented herein.

FIG. 1 shows the biosynthetic pathway for the conversion of tryptophan to indigoid compounds, wherein NADPH represents nicotinamide adenine dinucleotide phosphate.

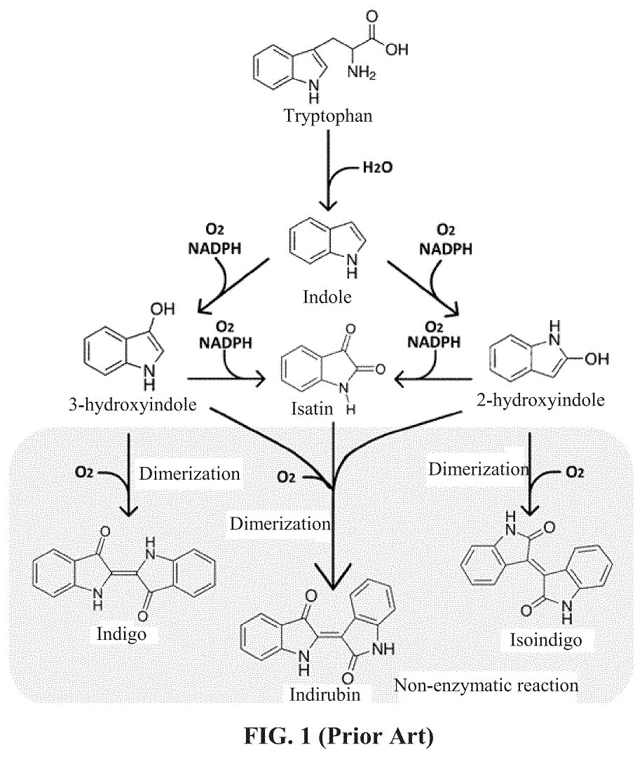

FIG. 2 shows mark comparison of the amino acid sequence differences between the wild-type flavin-containing monooxygenase from Corynebacterium glutamicum (cFMO) and the best mutant cFMO (cFMO M185V/V402M), wherein FAD-binding motif represents flavin adenine dinucleotide-binding motif, FMO-identifying motif represents flavin-containing monooxygenase-identifying motif, NADPH-binding motif represents nicotinamide adenine dinucleotide phosphate-binding motif, and the circled place is the mutation site.

FIG. 3 shows the effect of mutant cFMO on increasing indigo production, wherein the culture medium is LB medium containing 2 g/L tryptophan.

FIG. 4 shows a synthetic pathway utilizing cysteine to block 3-hydroxyindole dimerization and promote indirubin production.

FIG. 5 shows the effect of mutant cFMO on increasing indirubin production, wherein the culture medium is LB medium containing 2 g/L tryptophan and 3 mM cysteine.

FIG. 6 shows the effect regarding different mutation combinations of amino acid residues 185 and/or 402 on mutant cFMO on increasing indigo production, wherein the culture medium is LB medium containing 2 g/L tryptophan.

FIG. 7 shows the effect regarding different mutation combinations of amino acid residues 185 and/or 402 on mutant cFMO on increasing indirubin production, wherein the culture medium is LB medium containing 2 g/L tryptophan and 3 mM cysteine.

FIG. 8 shows the analytic result of Futile NADPH oxidase activity of mutant cFMO without reactant indole.

FIG. 9 shows the effect regarding different mutation combinations (mutant cFMO (M185VN402M), mutant cFMO (M185LN402A), mutant cFMO (M185V/V402R), mutant cFMO (M185FN402S), mutant cFMO (M185AN402R), mutant cFMO (M185YN402R), mutant cFMO (M185V/V402S), and mutant cFMO (M185LN402R)) of amino acid residues 185 and/or 402 on mutant cFMO on increasing indigo production over a long period of time.

FIG. 10 shows the effect regarding different mutation combinations (mutant cFMO (M185VN402M), mutant cFMO (M185LN402A), mutant cFMO (M185V/V402R), mutant cFMO (M185FN402S), mutant cFMO (M185AN402R), mutant cFMO (M185YN402R), mutant cFMO (M185V/V402S), and mutant cFMO (M185LN402R)) of amino acid residues 185 and/or 402 on mutant cFMO on increasing indirubin production over a long period of time.

### DETAILED DESCRIPTION OF THE PREFERRED EMBODIMENT

In the following detailed description of the embodiments of the present invention, reference is made to the accompanying drawings, which are shown to illustrate the specific embodiments in which the present disclosure may be practiced. These embodiments are provided to enable those skilled in the art to practice the present disclosure. It is understood that other embodiments may be used and that changes can be made to the embodiments without departing from the scope of the present invention. The following description is therefore not to be considered as limiting the scope of the present invention.

### Definition

As used herein, the data provided represent experimental values that can vary within a range of ±20%, preferably within ±10%, and most preferably within ±5%.

The method for increasing production of an indigoid compound (e.g., indigo and indirubin) of the present invention comprises the following steps: (a) mutating a wild-type flavin-containing monooxygenase (FMO) to a mutant FMO expressed in Escherichia coli; and (b) culturing the Escherichia coli in a bacterial culture medium comprising tryptophan to allow the mutant FMO to interact with tryptophan for a predetermined time to convert the tryptophan into the indigoid compound; wherein compared to the wild-type FMO, the mutant FMO increases production of the indigoid compound.

In the following examples, the term “bFMO” refers to FMO derived from bacteria; and cFMO is one of bFMO, derived from Corynebacterium glutamicum.

As used herein, the term wild-type FMO is derived from Corynebacterium glutamicum, also known as cFMO-WT. The mutant FMO is also derived from Corynebacterium glutamicum, also known as cFMO-Mutant.

In the following examples, the mutant cFMO comprises at least one mutation site. The at least one mutation site is located at amino acid residue 185 of the mutant cFMO, amino acid residue 402 of the mutant cFMO, or a combination thereof.

In the following examples, the amino acid residue 185 of the mutant cFMO is formed by mutating methionine residue 185 of the wild-type cFMO to a valine residue, hereinafter referred to as cFMO M185V.

In the following examples, the amino acid residue 402 of the mutant cFMO is formed by mutating valine residue 402 of the wild-type cFMO to a methionine residue, hereinafter referred to as cFMO V402M.

In the following examples, the amino acid residue 185 of the mutant cFMO is formed by mutating methionine residue 185 of the wild-type cFMO to a leucine residue, hereinafter referred to as cFMO M185L.

In the following examples, the amino acid residue 185 of the mutant cFMO is formed by mutating methionine residue 185 of the wild-type cFMO to a tyrosine residue, hereinafter referred to as cFMO M185Y.

In the following examples, the amino acid residue 185 of the mutant cFMO is formed by mutating methionine residue 185 of the wild-type cFMO to a phenylalanine residue, hereinafter referred to as cFMO M185F.

In the following examples, the amino acid residue 185 of the mutant cFMO is formed by mutating methionine residue 185 of the wild-type cFMO to an alanine residue, hereinafter referred to as cFMO M185A.

In the following examples, the amino acid residue 402 of the mutant cFMO is formed by mutating valine residue 402 of the wild-type cFMO to an alanine residue, hereinafter referred to as cFMO V402A.

In the following examples, the amino acid residue 402 of the mutant cFMO is formed by mutating valine residue 402 of the wild-type cFMO to an arginine residue, hereinafter referred to as cFMO V402R.

In the following examples, the amino acid residue 402 of the mutant cFMO is formed by mutating valine residue 402 of the wild-type cFMO to a serine residue, hereinafter referred to as cFMO V402S.

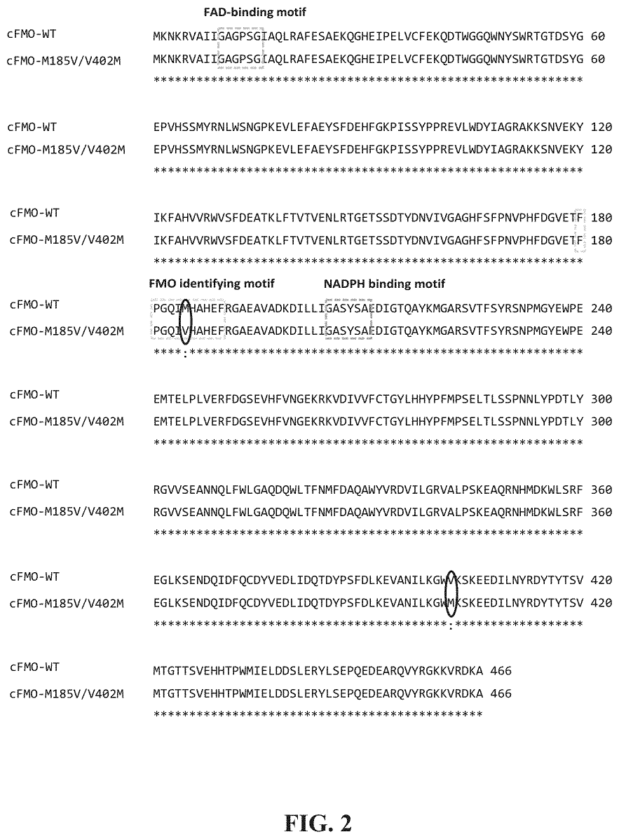

In the following examples, the polypeptide of the mutant cFMO comprises an amino acid sequence selected from the group consisting of: SEQ ID NO:1, SEQ ID NO:2, and SEQ ID NO:3. The mark comparison of the amino acid sequence differences between the wild-type cFMO and the best mutant cFMO (cFMO M185V/V402M) is shown in FIG. 2, wherein FAD-binding motif represents flavin adenine dinucleotide-binding motif, FMO-identifying motif represents flavin-containing monooxygenase-identifying motif, NADPH-binding motif represents nicotinamide adenine dinucleotide phosphate-binding motif, and the circled place is the mutation site.

Most of the chemicals, reagents and standards for high performance liquid chromatography (HPLC) used in the following examples are purchased from Sigma-Aldrich (Saint Louis, Mo.). The KOD DNA polymerase is purchased from EMD Chemicals (San Diego, Calif.). Primers used in all experiments are purchased from IDT (Singapore). The prepared plasmid is sequenced and confirmed by MISSION BIOTECH CO., LTD.

In the following examples, plasmid preparation and amplification and experimental strains for the production of indigo and indirubin are all Escherichia coli XL-1 Blue (Stratagene, CA). XL-1 belongs to the K strain in Escherichia coli, and the mutant hsdR17 makes the Escherichia coli K strain endonuclease system ineffective, and enhances the stability of exogenous DNA and the plasmid concentration after Miniprep. In addition, there are also mutations to endA and recA in XL-1 Blue, and mutation of endA can increase the concentration of plasmid after a small amount of extraction. Mutation of recA can increase the stability of plasmids implanted with XL-1 Blue and reduce the occurrence of mutations. Heat shock was used for plasmid production, and the plasmid was implanted into XL-1 Blue for plasmid amplification. Regarding plasmid for production of indigo and indirubin, electroporation was used to implant the plasmids into XL-1 Blue, which had been prepared as competent cells, for production experiments.

In the following examples, the plasmids were constructed according to the Gibson isothermal DNA assembly method (see Gibson, D. G., et al., Enzymatic assembly of DNA molecules up to several hundred kilobases. Nature methods, 2009. 6(5): p. 343-345). Each gene fragment to be assembled was prepared and purified by polymerase chain reaction (PCR), and then AMM reagents (exonuclease, polymerase and ligase) were used to assemble plasmids and sent to MISSION BIOTECH CO., LTD. for sequencing confirmation. After confirmation, the gene fragment was transfected into XL-1 Blue strain by heat shock method and stored at −80° C. The plasmids were stored at −20° C. The strains and plasmids used in the experiment are shown in Table 1, and the primers used are shown in Table 2.

TABLE 1

| | Genotype |
|---|---|
| Strain | |
| XL-1 | recA1 endA1 gyrA96 thi-1 hsdR17 supE44 relA1 lac [F′ proAB |
| | lacI^{q} |
| Blue | ZΔM15 Tn10 (Tet^{R})] |
| Plasmid | |
| pJY7 | P_{L}lacO¹:: fmo(CG); colE1 ori; Kan^{R} |
| pZY30 | P_{L}lacO¹:: fmo(CG) M185V/V402M; colE1 ori; Kan^{R} |
| pZY31 | P_{L}lacO¹:: fmo(CG) M185V; colE1 ori; Kan^{R} |
| pZY32 | P_{L}lacO¹:: fmo(CG) V402M; colE1 ori; Kan^{R} |
| pZY33 | P_{L}lacO¹:: fmo(CG); colE1 ori; Amp^{R} |
| pZY34 | P_{L}lacO¹:: fmo(CG) M185V/V402M; colE1 ori; Amp^{R} |
| pZY35 | P_{L}lacO¹:: fmo(CG) M185V; colE1 ori; Amp^{R} |
| pZY36 | P_{L}lacO¹:: fmo(CG) V402M; colE1 ori; Amp^{R} |
| pZY37 | P_{L}lacO¹:: fmo(CG) M185L/V402A; colE1 ori; Amp^{R} |
| pZY38 | P_{L}lacO¹:: fmo(CG) V402R; colE1 ori; Amp^{R} |
| pZY39 | P_{L}lacO¹:: fmo(CG) M185Y/V402R; colE1 ori; Amp^{R} |
| pZY40 | P_{L}lacO¹:: fmo(CG) M185V/V402S; colE1 ori; Amp^{R} |
| pZY41 | P_{L}lacO¹:: fmo(CG) M185L/V402R; colE1 ori; Amp^{R} |
| pZY42 | P_{L}lacO¹:: fmo(CG) (470AA); colE1 ori; Amp^{R} |
| pZY43 | P_{L}lacO¹:: fmo(CG) (470AA)T326S; colE1 ori; Amp^{R} |

TABLE 2

| Primer name | 5′→3′ sequence |
|---|---|
| pcFMO M185V | CCAGGTCAGATCGTGCATGCTCACGAGTTCCG (SEQ ID NO: 4) |
| SOE-f | |
| pcFMO M185V | CTCGTGAGCATGCACGATCTGACCTGGGAAAG (SEQ ID NO: 5) |
| SOE-r | |
| pcFMO V402M | TTGAAGGGCTGGATGAAGTCGAAGGAGGAGGA (SEQ ID NO: 6) |
| SOE-f | |
| pcFMO V402M | CTCCTTCGACTTCATCCAGCCCTTCAAGATATTCG (SEQ ID NO: 7) |
| SOE-r | |
| pcFMO (470AA) | TTCATTAAAGAGGAGAAAGGTACATGGAGATGGTTATGAAGAATA |
| his-f | AGCGCGTTGC (SEQ ID NO: 8) |
| pcFMO (466AA) | CATCACCATCACCATCACGGATCCGAAAACATGAAGAATAAGCGC |
| his-f | GTTGC (SEQ ID NO: 9) |
| pcFMO his-r | GAGCCTTTCGTTTTATTTGATGCCTCTAGATTAGGCTTTATCGCGGA |
| | CTTTCTTG (SEQ ID NO: 10) |
| pcFMO 185&402 | GGTGTGGAGACTTTCCCAGGTCAGATCNNKCATGCTCACGAGTTC |
| mutation 185-f | CGTGGTG (SEQ ID NO: 11), N represents A, C, G or T; K represents G or T |
| pcFMO 185&402 | GTTGAGGATATCCTCCTCCTTCGACTTMNNCCAGCCCTTCAAGATA |
| mutation 402-r | TTCGCG (SEQ ID NO: 12), M represents A or C; N represents A, C, G or T |

The LB medium contents used in the following examples are 10 g/L tryptone, 5 g/L yeast extract (YE) and 10 g/L NaCl.

In the following examples, random mutation techniques, including error prone PCR and site-directed random mutagenesis, were used to screen out the mutation sites of mutant cFMO. The Gene Morph II Random Mutagenesis Kit (Agilent) was used to perform error prone PCR, and PCR with an error prone polymerase was performed to generate random mutations in the target gene during the process of replication and amplification, so as to establish mutant gene library. In addition, double-site random mutation primers were designed to randomly mutate residues 185 and 402 of cFMO by PCR.

In following examples, the screening method for establishing double site random mutation strains was as follows. The ratio of the preparation ingredients is LB agar+0.2/1/2 g/L tryptophan+0.1 mM isopropyl-β-D-thiogalactopyranoside (IPTG) or +1/3/5/10 mM cysteine with appropriate antibiotics to find the screening conditions that the color difference between the strains can be seen. The final selection condition is LB agar+0.2 g/L tryptophan+0.1 mM IPTG with appropriate antibiotics for strain screening. Tryptophan is ingested into E. coli, tryptophanase produced by E. coli is used to convert tryptophan into indole, and cFMO is used to oxidize indole into indigo and indirubin.

In the following examples, the procedures regarding the analysis method of the product and reactant are as follows. The sampled bacterial solution was centrifuged at high speed, the supernatant was removed, the bacterial cells were redissolved in 1 mL of dimethyl sulfoxide (DMSO) solvent, and a bacteria breaker (MiniBeadBeater-16, model 607) was used. 3 rounds of 60-second bacterial disruption were performed to extract indigo and indirubin in the cells, and analysis was performed with high performance liquid chromatography (HPLC). The equipment for high performance liquid chromatograph is Shimadzu Prominence-i LC-2030C 3D with C18 column (5 μm, 4.6×250 mm), with methanol: pure water (75:25, v/v) as the mobile phase, and samples were analyzed at a flow rate of 1 mL/min and a column temperature of 25° C. First, a standard curve was made with 5 different concentrations of standards to obtain the slope, and the standard curve extrapolation method was used to calculate the concentration of indigo and indirubin. Indigo and indirubin samples were detected at spectral absorbance of 286 nm and 289 nm, respectively. In addition, for the analysis of tryptophan, the supernatant was sampled and analyzed by HPLC with methanol: pure water (50:50, v/v) as the mobile phase, the flow rate was 0.6 mL/min, and the spectral absorbance was 277 nm to detect tryptophan samples.

In the following examples, the procedures of enzyme purification are as follows. HisPur™ Ni-NTA purification kit (HisPur™ Ni-NTA Purification kit)(Thermo) was used for enzyme purification. The glycerol storage tube of XL-1 strain containing wild-type cFMO and mutant cFMO was taken from the −80° C. freezer, and the strain was inoculated to the tube containing 2 mL of LB and appropriate antibiotics (100 μg/mL ampicillin). Incubation was performed at 37° C., 250 rpm for 16-18 hours, and the resultant was inoculated to medium comprising 100 mL of LB and appropriate antibiotics (100 μg/mL penicillin) at a ratio of 1%. Incubation was performed at 37° C. and 250 rpm, and IPTG was added until OD₆₀₀ was between 0.6 and 0.8 to induce the target enzyme protein (i.e., FMO) highly expressed in the culture medium. A 30° C., 250 rpm incubator was used, and after culturing for 18-24 hours, the cells were collected by centrifugation and re-dissolved with 900 μL of appropriate buffer. The resultant was then mixed with 1 mL of 0.1 mm glass beads (Biospec) and subjected to 3 rounds of 30-second bacterial disruption using a bacteria breaker (MiniBeadBeater-16, model 607). In order to take out the products indigo and indirubin in E. coli, it is necessary to directly disrupt E. coli by the method of breaking bacteria to collect indigo and indirubin because E. coli cannot excrete indigo and indirubin from the cells. Centrifugation was performed at 4° C. to leave the supernatant, which contains all the water-soluble proteins in the bacterial cells. If the target enzyme protein (i.e., FMO) is tagged with His-tag, the next step is to perform washing and elution for protein purification according to the Thermo kit method, and the protein was finally collected by His-binding column purification. The protein concentration, such as FMO concentration, was detected by Bradford reagent.

### Example 1

Evaluation of Effect of Mutant cFMO on Increasing Production of Indigo

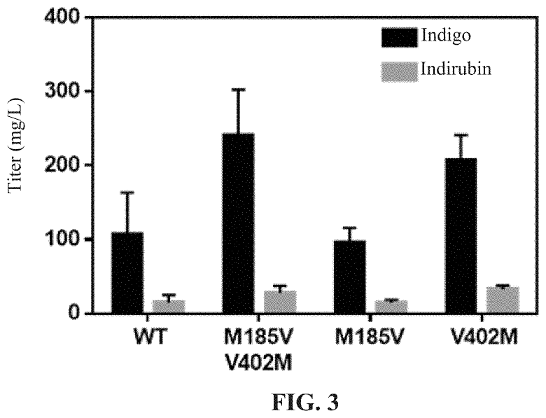

In this example, the indigo production of wild-type cFMO (cFMO-WT) and mutant cFMO (cFMO M185V/V402M) were compared to understand the production situation when the two mutation sites of cFMO M185V/V402M were individually single mutant. The strains cFMO M185V and cFMO V402M were subjected to a 24-hour production experiment, and the production was compared with two culture mediums. Four different enzymes (cFMO-WT, cFMO M185V/V402M, cFMO M185V and cFMO V402M) were produced in shake flasks with high copy plasmids in E. coli XL-1 Blue with 20 mL LB medium+2 g/L tryptophan for 24 hours, wherein high copy refers to the bacteria with a large amount of plasmids. Therefore, a large amount of enzyme cFMO can be relatively formed through transcription and translation for the production of indigo and indirubin. The result is shown in FIG. 3.

As shown in FIG. 3, about 250 mg/L indigo can be produced by mutant cFMO in E. coli XL-1 Blue using LB medium containing 2 g/L tryptophan for 24 hours in shake flasks. It can be seen that the mutation site 402 may be the key site that mainly affects the improvement of the production, but when the residue 185 is mutant with the residue 402 (i.e., cFMO M185VN402M), the production is indeed higher than either one mutation site.

### Example 2

Evaluation of Effect of Mutant cFMO on Increasing Production of Indirubin

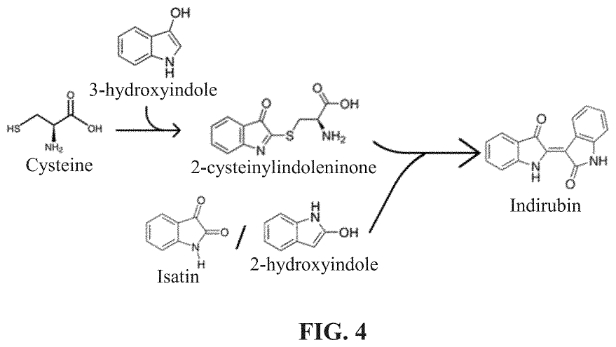

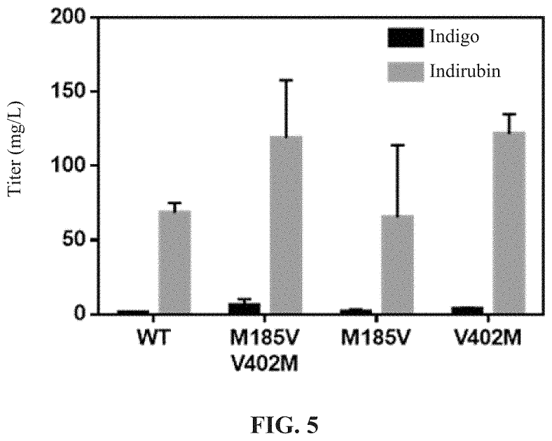

In this example, the reference (Han, G. H., et al., Enhanced indirubin production in recombinant Escherichia coli harboring a flavin-containing monooxygenase gene by cysteine supplementation. Journal of biotechnology, 2013. 164(2): p. 179-187; Kim, J., et al., Elucidating Cysteine Assisted Synthesis of Indirubin by a Flavin-Containing Monooxygenase. ACS Catalysis, 2019. 9(10): p. 9539-9544) pointed out that cysteine can promote synthesis of indirubin. Therefore, the production experiment was performed, see FIG. 4. Indirubin production by wild-type cFMO and mutant cFMO (cFMO M185V/V402M) was compared. Four different enzymes (cFMO-WT, cFMO M185VN402M, cFMO M185V and cFMO V402M) were produced in shake flasks with high copy plasmids in E. coli XL-1 Blue with 20 mL LB medium+2 g/L tryptophan+3 mM cysteine for 24 hours. The result is shown in FIG. 5.

As shown in FIG. 5, about 120 mg/L indirubin can be produced by culturing mutant cFMO in LB medium containing 2 g/L tryptophan and 3 mM cysteine for 24 hours in shake flasks, wherein cysteine is added in the culture medium of FIG. 5, and no cysteine is added in the culture medium of FIG. 3. According to the reference Kim, J., et al., Elucidating Cysteine Assisted Synthesis of Indirubin by a Flavin-Containing Monooxygenase. ACS Catalysis, 2019. 9(10): p. 9539-9544, it indicates that cysteine reacts with 3-hydroxyindole to form 2-cysteinylindoleninone, thereby inhibiting the spontaneous dimerization of 3-hydroxyindole to form indigo. Subsequently, 2-cysteinylindoleninone reacts with 2-hydroxyindole or isatin to form indirubin. It can be seen that the mutation site 402 may be the key site that mainly affects the improvement of the production, but when the residue 185 is mutant with the residue 402 (i.e., cFMO M185VN402M), the production is indeed higher than either one mutation site.

By comparing the results of the production of indigo and indirubin at single mutation site of the mutant cFMO M185V/V402M in Example 1 and this example, it can be seen that mutant cFMO M185V can produce about 100 mg/L indigo, and about 60 mg/L indirubin with 3 mM cysteine assistance; mutant cFMO V402M can produce about 200 mg/L indigo, and about 120 mg/L indirubin with 3 mM cysteine assistance. Although the yield at single mutation site of cFMO M185V is similar to that of cFMO-WT, when the two mutation sites M185V and V402M are combined, the yield can be significantly improved.

### Example 3

Evaluation of Effect Regarding Different Mutation Combinations of Amino Acid Residues 185 and/or 402 of Mutant cFMO on Increasing Production of Indigo and Indirubin

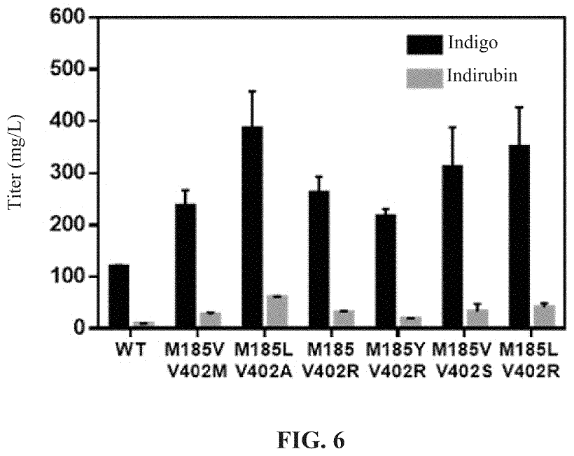

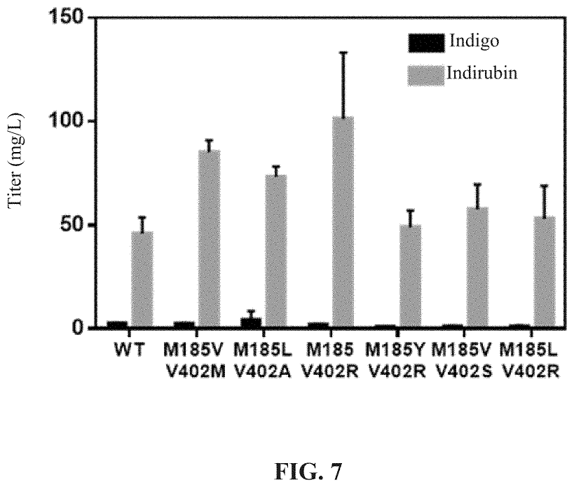

In this example, these two mutation sites (185 and 402) are further combined with other amino acid substitutions, and a darker blue candidate strain is initially screened with the LB agar plate. Screening is performed through the production experiment, and multiple mutant cFMO enzymes with better yield are screened out. The results are shown in FIGS. 6 and 7.

FIG. 6 shows the effect regarding different mutation combinations of amino acid residues 185 and/or 402 on mutant cFMO on increasing indigo production, and seven different enzymes (wild-type (WT) cFMO, mutant cFMO (M185VN402M), mutant cFMO (M185LN402A), mutant cFMO (M185V/V402R), mutant cFMO (M185YN402R), mutant cFMO (M185V/V402S), and mutant cFMO (M185L/V402R)) were produced in shake flasks with high copy plasmids in E. coli XL-1 Blue with 20 mL LB medium+2 g/L tryptophan for 24 hours, wherein mutant cFMO (M185LN402A) can produce about 500 mg/L indigo.

FIG. 7 shows the effect regarding different mutation combinations of amino acid residues 185 and/or 402 on mutant cFMO on increasing indirubin production, and seven different enzymes (wild-type (WT) cFMO, mutant cFMO (M185VN402M), mutant cFMO (M185LN402A), mutant cFMO (M185V/V402R), mutant cFMO (M185YN402R), mutant cFMO (M185V/V402S), and mutant cFMO (M185L/V402R)) were produced in shake flasks with high copy plasmids in E. coli XL-1 Blue with 20 mL LB medium+2 g/L tryptophan+3 mM cysteine for 24 hours. As shown in FIG. 7, mutant cFMO (M185/V402R) (SEQ ID NO:13) can produce about 140 mg/L indirubin with the assistance of 3 mM cysteine. The mutant cFMO (M185/V402R) has only a mutation at residue 402, while residue 185 has no mutation.

### Example 4

Futile Nicotinamide Adenine Dinucleotide Phosphate (NADPH) Oxidase Activity of Mutant cFMO without Reactant Indole

In this example, Futile NADPH oxidase activity of purified enzymes (cFMO-WT, cFMO M185V/V402M, cFMO M185L/V402A, cFMO M185V, and cFMO V402M) without reactant indole was compared, wherein without reactant indole represents that the enzyme cFMO would spontaneously react with NADPH without the reaction of indole, but originally cFMO reacts with indole and NADPH to generate indigo and indirubin.

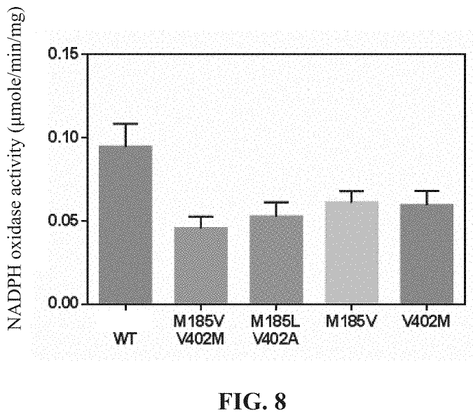

NADPH oxidase activity refers to that NADPH can carry out redox reaction with FMO to form NADP⁺ and produce H₂O₂, and the aforementioned reaction is an unpleasant competitive reaction when FMO produces indigoid compounds. This type of reaction also occurs with other NADPH oxidases, which reacts with oxygen in the presence of reactants to form H₂O₂, H₂O, and O₂⁻. The enzyme activity assay method is performed without reactant by adding potassium phosphate buffer (KPB) (0.1M, pH=8.0, KPB is prepared with 0.0935 M K₂HPO₄ and 0.0065 M KH₂PO₄), 0.1 mM ethylenediaminetetraacetic acid (EDTA), 2 μM flavin adenine dinucleotide (FAD), and 0.1/0.2 mM NADPH and reacting with enzymes for 30 minutes, and the decrease in absorbance at 340 nm was simultaneously measured. The result is shown in FIG. 8.

FIG. 8 shows the analytic result of Futile NADPH oxidase activity of mutant cFMO without reactant indole. Since NADPH, in addition to being an important cofactor for the formation of indigo and indirubin from cFMO, also competes with indole for reaction sites and undergoes redox reactions with water (competitive reactions). Therefore, the reference is referred in this example (see Jung, H. S., et al., Protein Engineering of Flavin-containing Monooxygenase from Corynebacterium glutamicum for Improved Production of Indigo and Indirubin. Journal of Life Science, 2018. 28(6): p. 656-662) to measure the NADPH oxidase activity without reactant indole (Futile NADPH oxidase activity). The activity of cFMO M185VN402M is 0.48 times more than that of cFMO-WT, and the activity of cFMO M185LN402A is 0.55 times more than that of cFMO-WT (see FIG. 8). That is, the NADPH oxidase activity of the mutant cFMO is lower than that of the wild-type cFMO, which means that the mutant would not perform competition reaction in which FMO spontaneously reacts with NADPH. Therefore, the reduction of NADPH oxidase activity may be related to the increase in the production of indigoid compounds of the mutant cFMO, so the mutant cFMO reduces the competition reaction, which is beneficial to the generation of the target product.

### Example 5

Evaluation of Effect Regarding Different Mutation Combinations of Amino Acid Residues 185 and/or 402 on Mutant cFMO on Increasing Indigo and Indirubin Production Over a Long Period of Time

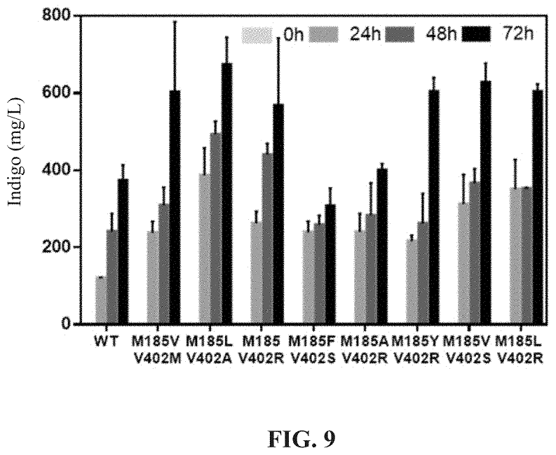

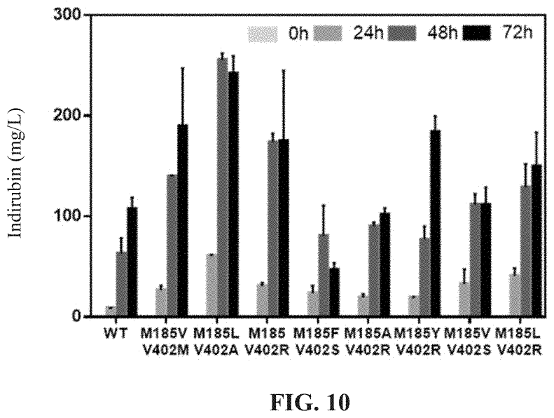

In this example, a three-day long-term production experiment for each mutant cFMO and wild-type cFMO was performed and compared. The results are shown in FIGS. 9 and 10.

FIG. 9 shows the effect regarding different mutation combinations (mutant cFMO (M185V/V402M), mutant cFMO (M185L/V402A), mutant cFMO (M185V/V402R), mutant cFMO (M185F/V402S), mutant cFMO (M185A/V402R), mutant cFMO (M185Y/V402R), mutant cFMO (M185V/V402S), and mutant cFMO (M185L/V402R)) of amino acid residues 185 and/or 402 on mutant cFMO on increasing indigo production over a long period of time. The long-term indigo production result for mutant combinations was compared. Seven different enzymes were produced in shake flasks with high copy plasmids in E. coli XL-1 Blue with 20 mL LB medium+2 g/L tryptophan for 24-72 hours.

FIG. 10 shows the effect regarding different mutation combinations (mutant cFMO (M185VN402M), mutant cFMO (M185LN402A), mutant cFMO (M185V/V402R), mutant cFMO (M185FN402S), mutant cFMO (M185AN402R), mutant cFMO (M185YN402R), mutant cFMO (M185V/V402S), and mutant cFMO (M185LN402R)) of amino acid residues 185 and/or 402 on mutant cFMO on increasing indirubin production over a long period of time. The long-term indirubin production result for mutant combinations was compared. Seven different enzymes were produced in shake flasks with high copy plasmids in E. coli XL-1 Blue with 20 mL LB medium+2 g/L tryptophan for 24-72 hours.

As shown in FIGS. 9 and 10, mutant cFMO (M185L/V402A) (SEQ ID NO:14) is the enzyme with the best three-day continuous production capacity, producing 670 mg/L indigo (see FIG. 9) and 240 mg/L indirubin (see FIG. 10) within 72 hours.

In summary, the present invention has the following effects. The mutant FMO can be screened out by using random mutation technology and screening of enzymes through protein engineering technology, and it is found that the combination of amino acid residues at specific positions in the amino acid sequences of the mutant FMO can effectively increase production of indigoid compounds, so that the yield of indigo and indirubin is more than 2 to 3 times higher than that of strains expressing wild-type FMO.

Although the present invention has been described with reference to the preferred embodiments, it will be apparent to those skilled in the art that a variety of modifications and changes in form and detail may be made without departing from the scope of the present invention defined by the appended claims.

What is claimed is:

1. A method for increasing production of an indigoid compound, comprising the following steps:
(a) mutating a wild-type flavin-containing monooxygenase (FMO) to a mutant FMO expressed in Escherichia coli; and
(b) culturing the Escherichia coli in a bacterial culture medium comprising tryptophan to allow the mutant FMO to interact with tryptophan for a predetermined time to convert the tryptophan into the indigoid compound;
wherein compared to the wild-type FMO, the mutant FMO increases production of the indigoid compound.

2. The method according to claim 1, wherein both the wild-type FMO and the mutant FMO are from Corynebacterium glutamicum.

3. The method according to claim 2, wherein the mutant FMO comprises at least one mutation site.

4. The method according to claim 3, wherein the at least one mutation site is located at amino acid residue 185 of the mutant FMO, amino acid residue 402 of the mutant FMO, or a combination thereof.

5. The method according to claim 4, wherein the amino acid residue 185 of the mutant FMO is formed by mutating methionine residue 185 of the wild-type FMO to a valine residue.

6. The method according to claim 4, wherein the amino acid residue 402 of the mutant FMO is formed by mutating valine residue 402 of the wild-type FMO to a methionine residue.

7. The method according to claim 5, wherein the amino acid residue 402 of the mutant FMO is formed by mutating valine residue 402 of the wild-type FMO to a methionine residue.

8. The method according to claim 4, wherein the amino acid residue 402 of the mutant FMO is formed by mutating valine residue 402 of the wild-type FMO to the arginine residue.

9. The method according to claim 4, wherein the amino acid residue 185 of the mutant FMO is formed by mutating methionine residue 185 of the wild-type FMO to a leucine residue, and the amino acid residue 402 of the mutant FMO is formed by mutating valine residue 402 of the wild-type FMO to an alanine residue.

10. The method according to claim 1, wherein the indigoid compound is selected from the group consisting of: indigo, indirubin, and a combination thereof.

11. The method according to claim 9, wherein the bacterial culture medium comprising tryptophan further comprises cysteine.

12. The method according to claim 1, wherein the mutant FMO is expressed in Escherichia coli XL-1 Blue.

13. The method according to claim 1, wherein in step (b), the predetermined time is at least 24 hours.

14. A recombinant polypeptide for increasing production of an indigoid compound, comprising an amino acid sequence selected from the group consisting of: SEQ ID NO:1, SEQ ID NO:2, SEQ ID NO:3, SEQ ID NO:13, and SEQ ID NO:14.

15. The recombinant polypeptide according to claim 13, which is a polypeptide of a mutant FMO.

16. The recombinant polypeptide according to claim 14, wherein the amino acid sequence of SEQ ID NO:1 is formed by mutating methionine residue 185 of a wild-type FMO to a valine residue.

17. The recombinant polypeptide according to claim 14, wherein the amino acid sequence of SEQ ID NO:2 is formed by mutating valine residue 402 of a wild-type FMO to a methionine residue.

18. The recombinant polypeptide according to claim 14, wherein the amino acid sequence of SEQ ID NO:3 is formed by mutating methionine residue 185 of a wild-type FMO to a valine residue, and by mutating valine residue 402 of the wild-type FMO to a methionine residue.

19. The recombinant polypeptide according to claim 14, wherein the amino acid sequence of SEQ ID NO:13 is formed by mutating valine residue 402 of the wild-type FMO to the arginine residue.

20. The recombinant polypeptide according to claim 14, wherein the amino acid sequence of SEQ ID NO:14 is formed by mutating methionine residue 185 of a wild-type FMO to a leucine residue, and by mutating valine residue 402 of the wild-type FMO to an alanine residue.
